(19) United States
(12) Patent Application Publication
(10) Pub. No.: US 2014/0114213 A1
(43) Pub. Date: Apr. 24, 2014

(54) POSTURAL SWAY AND STABILITY MEASUREMENT AND THERAPEUTIC DEVICE AND RELATED METHODS

(71) Applicant: East Carolina University, Greenville, NC (US)

(72) Inventors: Leslie K. Allison, Winston-Salem, NC (US); Ryan Matthew Shoaf, Harrisonburg, VA (US); Melissa VanderLinde, Mooresville, NC (US)

(73) Assignee: East Carolina University, Greenville, NC (US)

(21) Appl. No.: 14/057,345

(22) Filed: Oct. 18, 2013

Related U.S. Application Data
(60) Provisional application No. 61/716,083, filed on Oct. 19, 2012.

(51) Int. Cl. A61B 5/11

(52) U.S. Cl. CPC A61B 5/112
USPC 600/595

(57) ABSTRACT

A postural and sway stability measurement device includes a slidable support that is configured to be mounted on a base such that the slidable support is movable between an initial position and a plurality of a maximum sway positions; and a patient sway unit mounted to the slidable support. The patient sway unit is configured to interact with a patient such that when the patient leans in a direction, the patient sway unit moves the slidable support from the initial position to one of the maximum sway positions to thereby measure an amount of maximum sway for the patient.

### RELATED APPLICATIONS

This application claims priority to U.S. Provisional Application Ser. No. 61/716,083 filed Oct. 19, 2012, the disclosure of which is incorporated by reference in its entirety.

### FIELD OF THE INVENTION

The present invention relates to a postural sway and stability measurement and therapeutic device and related methods.

### BACKGROUND

Physically unstable individuals, such as elderly persons over 65-75 years of age, may suffer a fall if the individual's point of balance, or center of gravity, moves outside their base of support. The base of support is generally outlined by area defined by the individual's feet on the floor. In order to study the stability of various patient groups, physical therapists generally use two methods to identify when a patient has exceeded, or is danger of exceeding, the limits of their stability.

One method currently in use is called the Limits of Stability test, which provides physical therapists with measurements taken when the subject intentionally displaces his/her center of gravity by leaning in a direction without losing balance. A Limits of Stability typically involves a base support that includes sensors that are placed on a floor or mat to detect the subject's center of gravity. The Limits of. Stability testing equipment may be relatively expensive and is generally only deployed in a clinical setting.

A less expensive alternative to the Limits of Stability testing equipment is the Multi-Directional Functional Reach test. In the Multi-Directional Functional Reach test, the subject's reach distance is measured. The Multi-Directional Functional Reach test, however, is not as accurate as the Limits of Stability test, and may not be suitable for older patients.

### SUMMARY OF EMBODIMENTS OF THE INVENTION

In some embodiments, a postural and sway stability measurement device includes a slidable support that is configured to be mounted on a base such that the slidable support is movable between an initial position and a plurality of a maximum sway positions; and a patient sway unit mounted to the slidable support. The patient sway unit is configured to interact with a patient such that, when the patient leans in a direction, the patient sway unit moves the slidable support from the initial position to one of the maximum sway positions to thereby measure an amount of maximum sway for the patient.

In some embodiments, the patient sway unit comprises an elongated member and the slidable support comprises at least two slidable members that are spaced apart along the elongated member. The at least two slidable members may define a patient test region in which the patient is positioned so as to interact with the patient sway unit. The base may include a pair of parallel support bars and the at least two slidable members are configured to be mounted on respected ones of the pair of parallel support bars. The at least two slidable members may include a generally cylindrical shape.

In some embodiments, the base comprises an elongated member having at least one fastener configured to connect the base to a furniture edge.

In some embodiments, the patient sway unit is adjustably connected to the slidable member such that a height of the patient sway unit is adjustable in response to a patient height.

In some embodiments, the patient sway unit comprises a front portion that is configured to interact with a side of the patient and a back portion that is configured to interact with another opposing side of the patient such that the slidable member moves in opposing directions to determine a sway distance in the opposing directions.

In some embodiments, the device includes a sway sensor configured to detect a sway distance when the patient sway unit is moved to a maximum sway position. The sway sensor may include an optical sensor and/or an accelerometer.

In some embodiments, the device includes a sway feedback module that is configured to provide a feedback indication to the user. The sway feedback module may be in communication with a user interface and may be configured to trigger an output to the user interface when the sway distance satisfies a predetermined value.

In some embodiments, methods for measuring postural and sway stability include providing a postural and sway stability measurement device. The device includes a slidable support that is configured to be mounted on a base such that the slidable support is movable between an initial position and a plurality of a maximum sway positions. A patient sway unit is mounted to the slidable support. The patient sway unit is configured to interact with a patient such that when the patient leans in a direction, the patient sway unit moves the slidable support from the initial position to one of the maximum sway positions to thereby measure an amount of maximum sway for the patient. The method includes measuring a sway distance in at least one direction based on a sliding movement of the patient sway unit and/or slidably support responsive to movement of a subject, and optionally determining a likelihood of falling based on the sway distance.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a perspective view of a sway and stability measurement device according to some embodiments.

FIG. 2 is a perspective view of the sway and stability measurement device of FIG. 1 shown in use on a pair of parallel support bars.

FIG. 3 is a perspective view of the sway and stability measurement device of FIG. 1 in use with a patient detecting forward sway.

FIG. 4 is a perspective view of the sway and stability measurement device of FIG. 1 in use with a patient detecting backward sway.

FIG. 5 is a perspective view of the sway and stability measurement device of FIG. 1 in use with a patient detecting sideward sway.

FIG. 6 is a perspective view of a sway and stability measurement device according to some embodiments that include a fastener for connecting the device to an object.

FIG. 7 is a top view of the device of FIG. 6.

FIG. 8 is a perspective view of a sway and stability measurement device according to some embodiments in which measurements may be obtained in two, opposing directions.

FIG. 9 is a schematic diagram illustrating methods, systems and computer program products according to some embdodiments.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer- implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

The computer-usable or computer-readable medium may be, for example but not limited to, an electronic, optical, electromagnetic, infrared, or semiconductor system, apparatus, or device. More specific examples (a non-exhaustive list) of the computer readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

As used herein, the term “sway” refers to a measurement or distance that a patient or user moves away from his or her center of gravity or base of support.

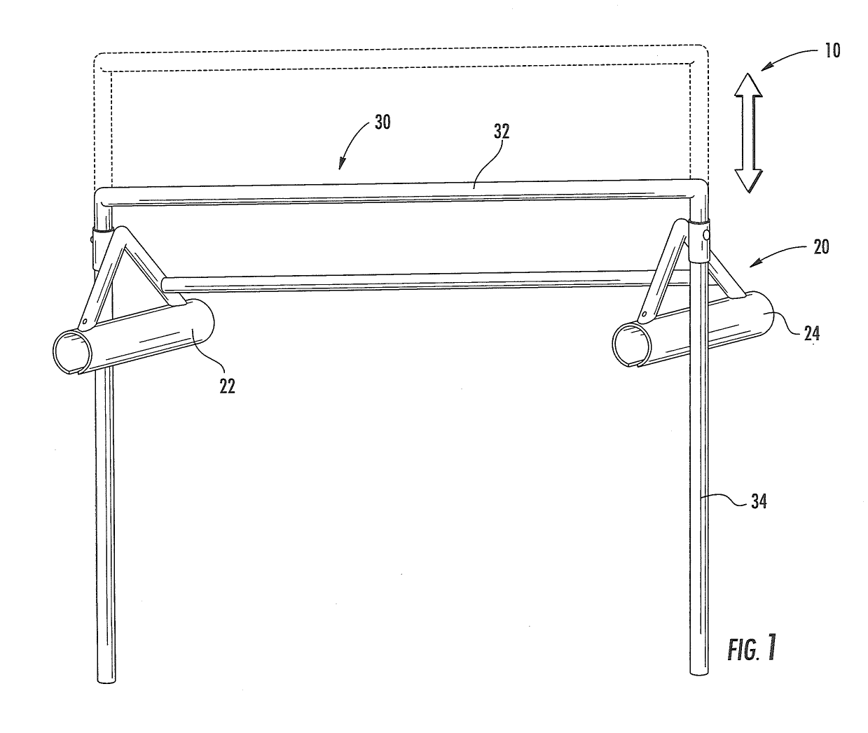

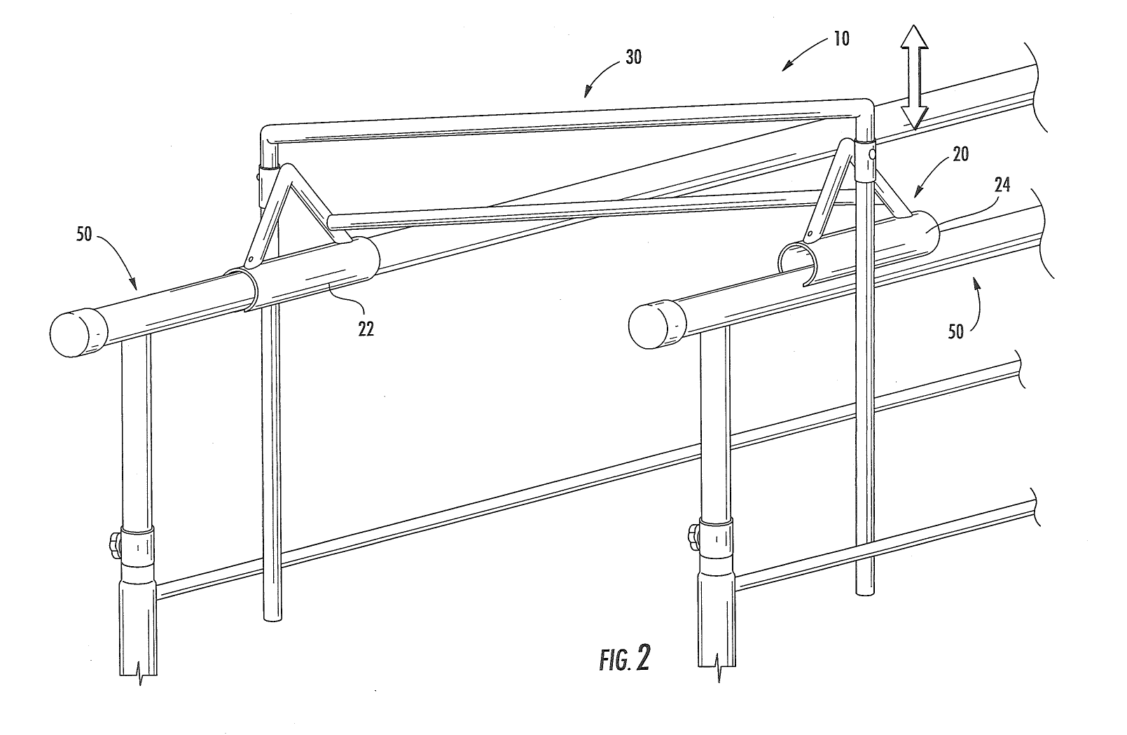

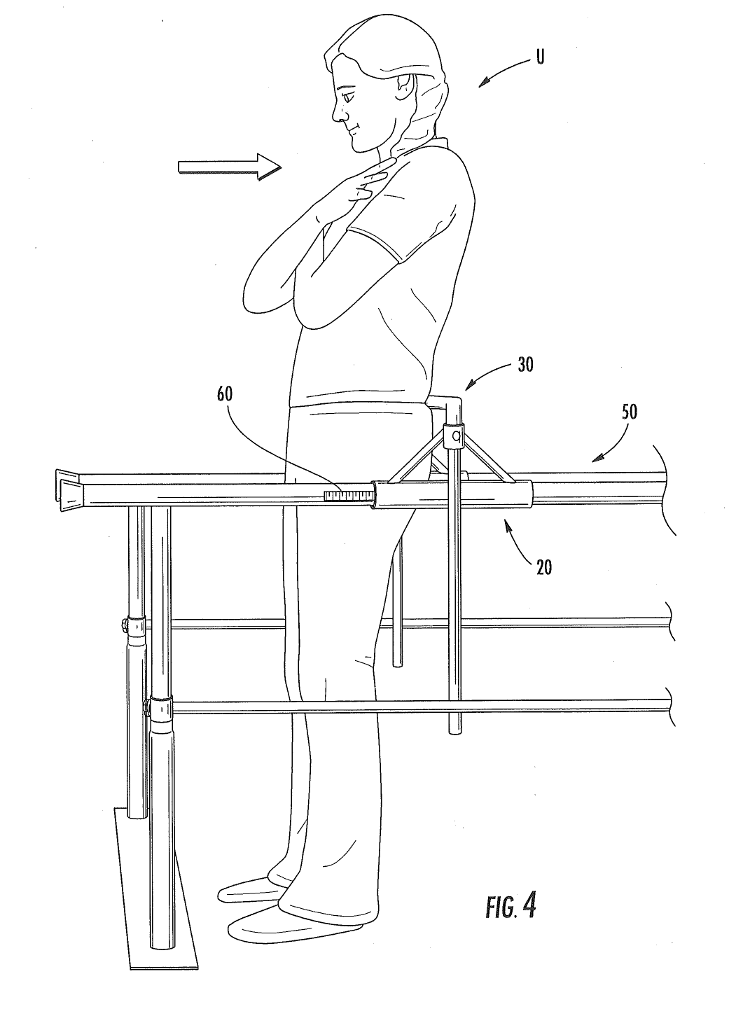

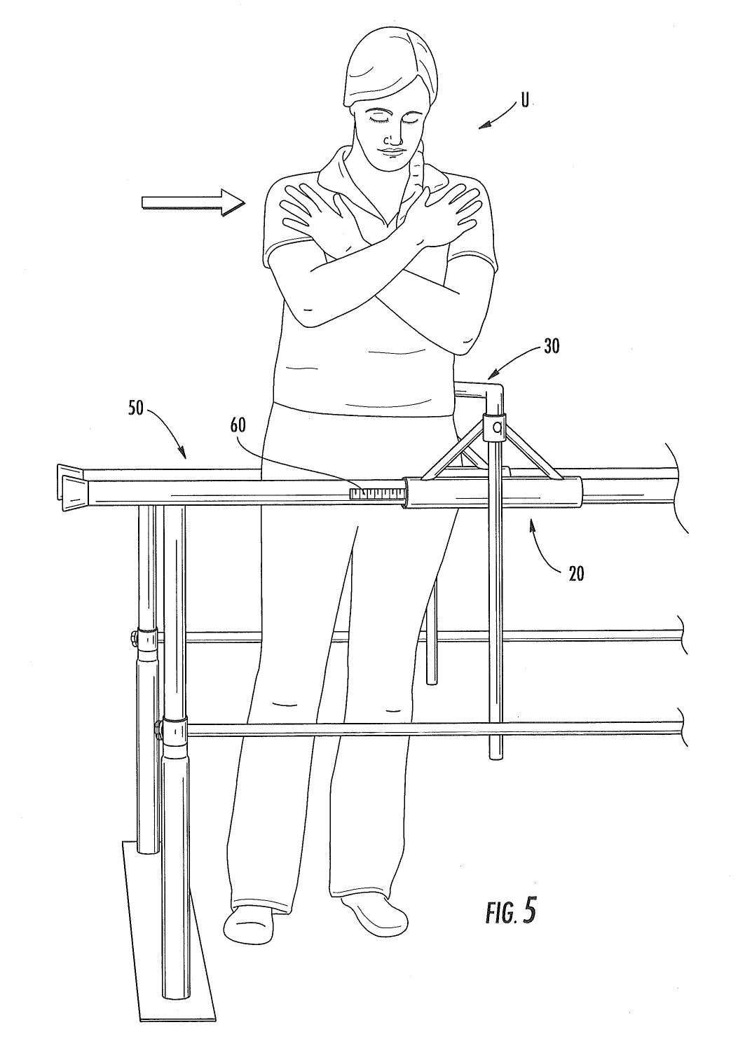

As illustrated in FIGS. 1-5, a postural sway and stability measurement device 10 according to some embodiments includes a slidable support 20 and a patient sway unit 30. The slidable support 20 includes two, generally cylindrical slidable members 22, 24 that are configured to slidably connect to a base, such as a pair of parallel support bars 50 (FIGS. 2-5). The patient sway unit 30 has a horizontal bar 32 and telescoping side supports 34 such that the height of the horizontal bar 32 is adjustable with respect to the slidable members 22, 24 with a telescoping action.

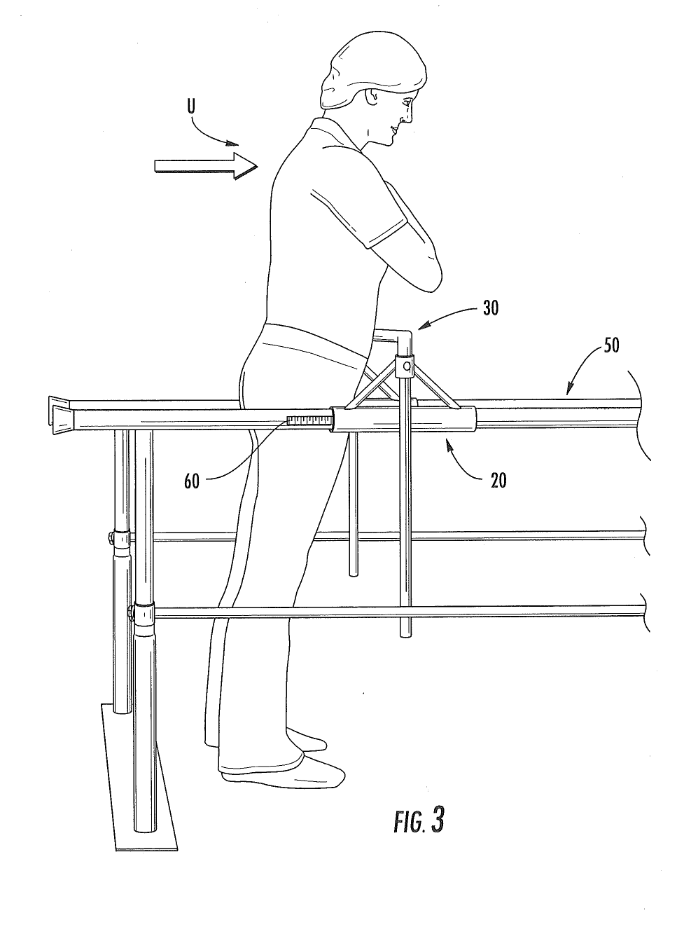

As illustrated in FIGS. 3-5, the parallel bars 50 may include a distance measurement indicator or ruler 60. In this configuration, the slidable members 22, 24 define a patient test region between the slidable members 22, 24 in which a user U may be positioned so as to interact with the patient sway unit 30. When the user U is upright, the ruler 60 is hidden within the cylindrical members 22, 24. When the user U leans in a direction towards the patient sway unit 30, the user U moves the patient sway unit 30 so that the slidable support 20 moves along the parallel bars 50. The amount of sway may be visibly determined by viewing the position of the cylindrical members 22, 24 on the ruler 60. As shown in FIGS. 3-5, the sway distances of a user U may be determined in a forward, backward or side-to-side. One or more of the sway distances of a user U may be used for diagnostic and/or therapeutic purposes. For example, a user's likelihood of falling may be determined based on a database of actual clinical experience, e.g., using a regression analysis of patient data to determine a probability of falling. A user's falling likelihood and/or sway distances may by used to prescribe various therapeutic exercises. In some embodiments, the therapeutic exercise may include repetitions of swaying using the device 10 to increase a sway distance.

It should be understood that, although a ruler 60 is illustrated, other techniques for determining a sway distance may be used. For example, the ruler 60 may be omitted and a user may mark an initial distance and a maximum sway distance on the parallel bars 50 for later measurement. In some embodiments, a sway distance sensor may be added to the device 10 to detect the maximum sway distance. The sway distance sensor may be an optical sensor, such as a camera, that is positioned with respect to optical indicators on the parallel bars 50 or other object that is stationary. In some embodiments, the optical sensor may be a camera on a handheld device, such as a “smart” phone or other mobile terminal, that is affixed to the device 10, and the handheld device may communicate maximum sway distances to a user or user interface. Other examples of sway distance sensors include accelerometers, acoustic distance sensors, and laser-based distance sensors; however, any suitable distance or motion sensor may be used. For example, any position or distance sensor may be placed on the device 10 or on another stationary object, such as on the bars 50. The position of the device 10 in the initial or at rest position may be compared to the position of the device 10 when the patient moves the device 10 a sway distance. In one particular example, a laser-based distance sensor (e.g., a Bosch™ GLR225 from Robert Bosch LLC, Farmington Hills, Mich., USA) may be affixed to the device 10 and a mirror or other reflective surface may be affixed to the bar 50 in the line of site of the laser-based distance sensor to record the initial position and the sway position of the device 10.

In some embodiments, the sway distance may be communicated by a sensor to a computer processor or database and recorded for analysis and tracking.

The cylindrical slidable members 22, 24 may by any suitable shape for sliding along a surface, and may be configured to be slidably received on any desired surface. For example, the bars 50 may have a rectangular or other shaped cross-section, and the slidable members 22, 24 may be configured in a similar or cooperating shape corresponding to the cross-section of the bars 50.

In some embodiments, a feedback module is provided that may provide positive feedback that a user has reached a particular sway distance or goal. For example, a feedback module may be in communication with a feedback output device, such as a speaker or light, that provides a sound or visual feedback for a predetermined sway distance. A feedback module may be used to provide incentives to a user or to automatically communicate to the user when a desired sway distance has been reached. The feedback may be a sway distance measurement that is provided to a user and/or health care professional via a user interface or automatically conveyed to a printer that prints a report. The feedback may include dates, times, sway distances, graphs showing progress and the like.

Devices according to some embodiments may be formed of any suitable material, including elastomeric materials (plastic) or metal. Portions of the device may be formed of rigid materials; however, some portions of the device may be formed of flexible materials, e.g., to permit adjustments for the patient's size.

As illustrated in FIGS. 1-5, the device 10 is positioned on parallel bars 50, which may provide a potentially unstable patient with a stable object to grab if the patient should begin to fall during a sway measurement. However, additional patient supports, such as conventional gait belts used by physical therapists, may also be used for increased stability.

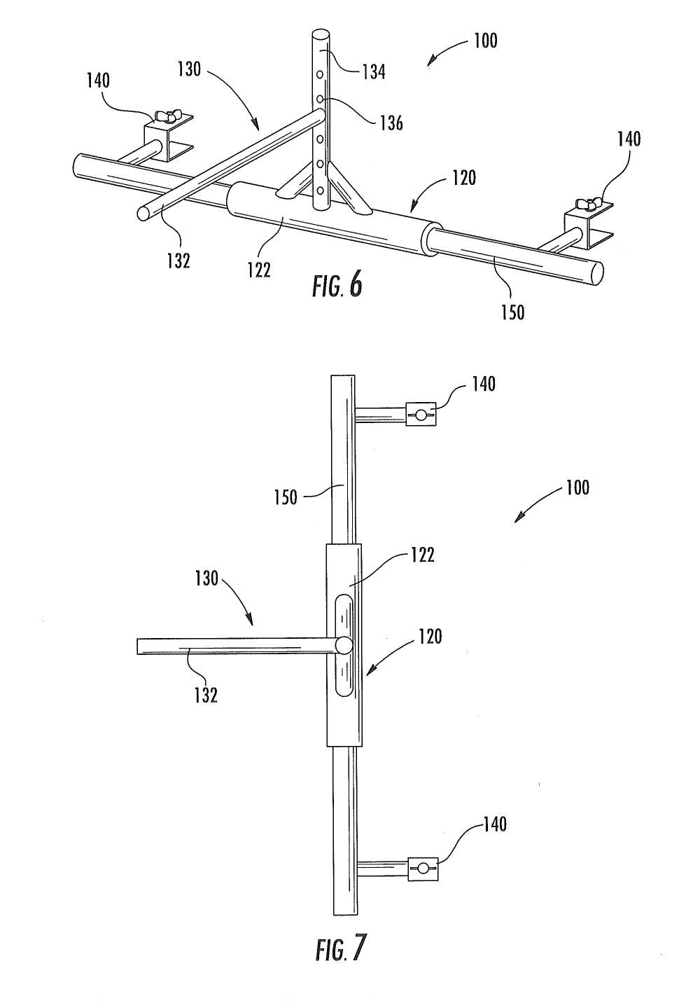

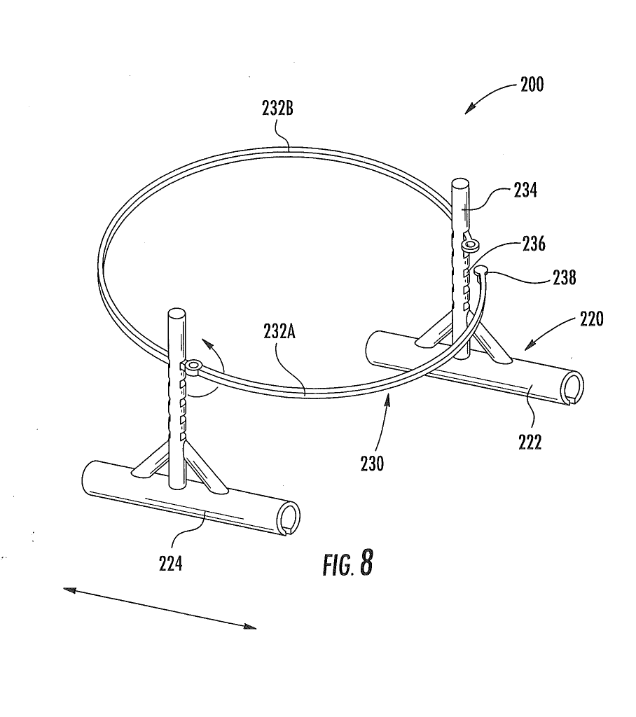

As illustrated, the parallel bars 50 and the slidable support 20 extend on either side of the patient. However, it should be understood that additional configurations are within the scope of the invention. For example, as illustrated in FIGS. 6-7, a sway and stability device 100 includes a slidable support 120 having a single slidable member 120 and a patient sway unit 130. The slidable member is configured to slidably connect to a base 150 that includes two fasteners or clamps 140. The patient sway unit 130 has a horizontal bar 132 and an adjustable side support 134 with apertures 136 such that the height of the horizontal bar 132 is adjustable by moving the bar 132 to different ones of the apertures 136.

Although the configuration illustrated in FIGS. 6-7 may be used in a clinical or therapeutic office setting, the device 100 may be particularly suitable for in-home use. The clamps 140 may be fastened to a table or countertop without requiring the use of parallel bars.

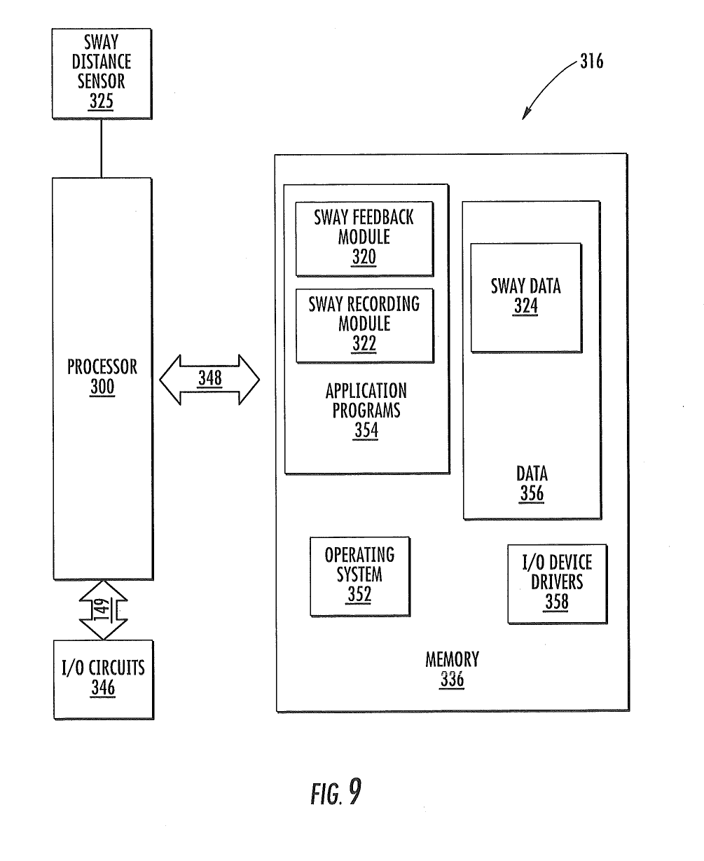

In addition, stability and sway measurement devices may be provided that are configured to measure a sway distance in two directions without requiring the repositioning of the patient. As illustrated in FIG. 8, a device 200 includes a slidable support 220 and a patient sway unit 230. The slidable support 220 includes two, generally cylindrical slidable members 222, 224 that are configured to slidably connect to a base, such as a pair of parallel support bar. The patient sway unit 230 has two semi-circular bars 232A, 232B that are configured to wrap around the patient such that the patient may move in a direction toward one of the bars 232A to measure a first sway distance, and the patient may move in a direction toward the other bar 232B to measure a second, opposite sway distance. The sway unit 230 includes side supports 234 with notches 236 for receiving the bars 232A, 232B at various adjustable heights. The bars 232A, 232B may also include a releasable clasp 238 that permits the patient to enter and exit the interior of the circle that is defined by the bars 232A, 232B. The bars 232A, 232B may also be hinged to permit upward/downward rotational movement. In some embodiments, the circumference defined by the bars 232A, 232B may be adjusted to account for patients of different sizes, e.g., by providing a telescoping feature or other mechanism for modifying the bars 232A, 232B.

FIG. 9 illustrates an exemplary data processing system 316 that may be included in devices operating in accordance with some embodiments of the present invention, to interact and/or control a sway distance sensor 325 on the postural sway and stability devices described herein and/or provide data and communications to a remote device. As illustrated in FIG. 9, a data processing system 316, which can be used to carry out or direct operations includes a processor 300, a memory 336 and input/output circuits 346. The data processing system can be incorporated in a portable communication device and/or other components of a network, such as a server. The processor 300 communicates with the memory 336 via an address/data bus 348 and communicates with the input/output circuits 346 via an address/data bus 349. The input/output circuits 346 can be used to transfer information between the memory (memory and/or storage media) 336 and another component, such a sway distance sensor 325. These components can be conventional components such as those used in many conventional data processing systems, which can be configured to operate as described herein.

In particular, the processor 300 can be a commercially available or custom microprocessor, microcontroller, digital signal processor or the like. The memory 336 can include any memory devices and/or storage media containing the software and data used to implement the functionality circuits or modules used in accordance with embodiments of the present invention. The memory 336 can include, but is not limited to, the following types of devices: cache, ROM, PROM, EPROM, EEPROM, flash memory, SRAM, DRAM and magnetic disk. In some embodiments of the present invention, the memory 336 can be a content addressable memory (CAM).

As further illustrated in FIG. 9, the memory (and/or storage media) 336 can include several categories of software and data used in the data processing system: an operating system 352; application programs 354; input/output device circuits 346; and data 356. As will be appreciated by those of skill in the art, the operating system 352 can be any operating system suitable for use with a data processing system, such as IBM®, OS/2®, AIX® or zOS® operating systems or Microsoft® Windows® operating systems Unix or Linux™. The input/output device circuits 346 typically include software routines accessed through the operating system 352 by the application program 354 to communicate with various devices. The application programs 354 are illustrative of the programs that implement the various features of the circuits and modules according to some embodiments of the present invention. Finally, the data 356 represents the static and dynamic data used by the application programs 354, the operating system 352 the input/output device circuits 346 and other software programs that can reside in the memory 336.

The data processing system 316 can include several modules, including a fluid sway feedback module 320, a sway recording module 322 and the like. In some embodiments, the sway feedback module 320 may receive sway data from the sway distance sensor 325 and provide feedback to the user, such as a sound or lights that indicate that a predetermined distance or goal has been achieved. The sway recording module 322 may record sway distances achieved by a user for analysis and/or diagnostic purposes. In some embodiments, the sway distances achieved by a user over time may be transmitted to a central database and combined with data from other users for analysis. For example, data from multiple users may be used to determine a likelihood of falling (e.g., predictive validity) and/or an effectiveness of treatment intervention. The modules can be configured as a single module or additional modules otherwise configured to implement the operations described herein. The data 356 can include sway data 324, for example, data that can be collected by the sway recording module 322.

While the present invention is illustrated with reference to the sway feedback module 320, the sway recording module 322 and the sway data 324 in FIG. 9, as will be appreciated by those of skill in the art, other configurations fall within the scope of the present invention. For example, rather than being an application program 354, these circuits and modules can also be incorporated into the operating system 352 or other such logical division of the data processing system. Furthermore, while the sway feedback module 320 and the sway recording module 322 in FIG. 9 are illustrated in a single data processing system, as will be appreciated by those of skill in the art, such functionality can be distributed across one or more data processing systems. Thus, the present invention should not be construed as limited to the configurations illustrated in FIG. 9, but can be provided by other arrangements and/or divisions of functions between data processing systems. For example, although FIG. 9 is illustrated as having various circuits and modules, one or more of these circuits or modules can be combined, or separated further, without departing from the scope of the present invention. In some embodiments, the operating system 352, programs 354 and data 356 may be provided as an integrated part of the sway distance sensor 325.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A postural and sway stability measurement device comprising:
a slidable support that is configured to be mounted on a base such that the slidable support is movable between an initial position and one or more a maximum sway positions; and
a patient sway unit mounted to the slidable support, the patient sway unit being configured to interact with a patient such that when the patient leans in a direction, the patient sway unit moves the slidable support from the initial position to one of the maximum sway positions to thereby measure an amount of maximum sway for the patient.

2. The postural and sway stability measurement device of claim 1, wherein the patient sway unit comprises an elongated member and the slidable support comprises at least two slidable members that are spaced apart along the elongated member.

3. The postural and sway stability measurement device of claim 2, wherein the at least two slidable members define a patient test region in which the patient is positioned so as to interact with the patient sway unit.

4. The postural and sway stability measurement device of claim 2, wherein the base comprises a pair of parallel support bars and the at least two slidable members are configured to be mounted on respected ones of the pair of parallel support bars.

5. The postural and sway stability measurement device of claim 4, wherein the at least two slidable members comprise a generally cylindrical shape.

6. The postural and sway stability measurement device of claim 1, wherein the base comprises an elongated member having at least one fastener configured to connect the base to a furniture edge.

7. postural and sway stability measurement device of claim 1, wherein the patient sway unit is adjustably connected to the slidable member such that a height of the patient sway unit is adjustable in response to a patient height.

8. The postural and sway stability measurement device of claim 1, wherein the patient sway unit comprises a front portion that is configured to interact with a side of the patient and a back portion that is configured to interact with another opposing side of the patient such that the slidable member moves in opposing directions to determine a sway distance in the opposing directions.

9. The postural and sway stability measurement device of claim 1, further comprising a sway sensor configured to detect a sway distance when the patient sway unit is moved to a maximum sway position.

10. The postural and sway stability measurement device of claim 9, wherein the sway sensor comprises an optical sensor and/or an accelerometer.

11. The postural and sway stability measurement device of claim 9, further comprising a sway feedback module that is configured to provide a feedback indication to the user.

12. The postural and sway stability measurement device of claim 11, wherein the sway feedback module is in communication with a user interface and is configured to trigger an output to the user interface when the sway distance satisfies a predetermined value.

13. A method for measuring postural and sway stability, the method comprising:
providing a postural and sway stability measurement device comprising:
a slidable support that is configured to be mounted on a base such that the slidable support is movable between an initial position and one or more maximum sway positions; and
a patient sway unit mounted to the slidable support, the patient sway unit being configured to interact with a patient such that when the patient leans in a direction, the patient sway unit moves the slidable support from the initial position to one of the maximum sway positions to thereby measure an amount of maximum sway for the patient;
measuring a sway distance in at least one direction based on a sliding movement of the patient sway unit and/or slidably support responsive to movement of a subject; and
optionally determining a likelihood of falling based on the sway distance.

14. The method of claim 13, wherein the device further comprises a sway sensor configured to detect a sway distance when the patient sway unit is moved to a maximum sway position, wherein measuring a sway distance comprises measuring a sway distance responsive to a sway distance output of the sway sensor.

15. The method of claim 14, wherein the sway sensor comprises an optical sensor and/or an accelerometer,

16. The method of claim 14, further comprising providing a feedback indication to the user responsive to the sway distance.

17. The method of claim 16, further comprising triggering an output to the user interface when the sway distance satisfies a predetermined value.

18. A computer program product for determining measuring postural and sway stability using a postural and sway stability measurement device comprising: a slidable support that is configured to be mounted on a base such that the slidable support is movable between an initial position and one or more maximum sway positions; a patient sway unit mounted to the slidable support, the patient sway unit being configured to interact with a patient such that when the patient leans in a direction, the patient sway unit moves the slidable support from the initial position to one of the maximum sway positions to thereby measure an amount of maximum sway for the patient; and a sway sensor configured to detect a sway distance when the patient sway unit is moved to a maximum sway position; the computer program product comprising a non-transitory computer readable storage medium having computer readable code embodied in the medium, the computer code comprising:
computer readable code configured to receive a signal from the sway sensor;
computer readable code configured to determine a sway distance responsive to the signal from the sway sensor; and
computer readable code configured to optionally determining a likelihood of falling based on the sway distance.

19. The computer program product of claim 18, further comprising computer readable code configured to provide a feedback indication to the user responsive to the sway distance.

20. The computer program product of claim 19, further comprising computer readable code configured to trigger an output to a user interface when the sway distance satisfies a predetermined value.
